(19) United States
(12) Patent Application Publication
(10) Pub. No.: US 2017/0311667 A1
(43) Pub. Date: Nov. 2, 2017

(54) SYSTEM AND METHOD FOR HAIR CONCEALMENT

(71) Applicants: Salvatore Passariello, North Reading, MA (US); Jane Morel, North Reading, MA (US)

(72) Inventors: Salvatore Passariello, North Reading, MA (US); Jane Morel, North Reading, MA (US)

(21) Appl. No.: 15/598,485

(22) Filed: May 18, 2017

Related U.S. Application Data
(63) Continuation-in-part of application No. 14/957,161, filed on Dec. 2, 2015.

(51) Int. Cl. A41G 5/00; B65D 1/02; A61K 8/98; A61Q 5/00; B65D 41/04

(52) U.S. Cl. CPC A41G 5/0013; B65D 41/04; B65D 1/0261; A61K 8/985; A61Q 5/00; B65D 1/023; A61K 2800/87

(57) ABSTRACT

Disclosed are a system, and method for fixation of hair fibers on a bald region on a human head. The method comprises pre-treating the bald region so that excess static electricity is removed from the bald region. The removal of static electricity improves fixation property the bald region. The method further includes, using a hair concealment device that is made up of a bottom and a top part. The top part is made up of metal and hence when a user touches metal portion with his hands, static electricity of the hair fibers is removed. This provides better fixation property to the human fibers and increases fixation to the bald region. The method further includes providing finishing to the bald region by spraying a soft gel to further improve fixation of hair to the bald region and natural human hair.

### TECHNICAL FIELD

The disclosed subject matter relates to a method and system for concealment of a balding region by using hair fibers.

### BACKGROUND

Hair loss, also known as alopecia or baldness, refers to a loss of hair from the head. Baldness can refer to general hair loss or male pattern hair loss. Male-pattern hair loss is hair loss that occurs due to an underlying 10 susceptibility of hair follicles to androgenic miniaturization. It is the most common cause of hair loss and will affect up to 70% of men and 40% of women at some point in their lifetimes. As can be seen, there is a need for a balding concealment method and apparatus.

### SUMMARY

According to aspects illustrated herein, there is provided a hair concealment system and method that helps in providing better fixation to hair fibers on a balding region.

According to an aspect of the invention, there is provided a hair concealment system that comprises an elongated bottle. The elongated body further has a top and a bottom part. The top and the bottom part define an open cavity to store a plurality of hair fibers. Further, the system includes a cover portion, wherein the cover portion is present at the top part of the elongated bottle. The system further includes a cap that is configured to be placed on the cover portion of the elongated bottle. The cover portion includes a plurality of openings, that have dimensions to allow at least one hair fiber. Further the plurality of hair fibers is released when the elongated bottle is stimulated.

According to another aspect of the invention, the present application includes a hair concealment system that includes an elongated bottle that further includes a top and a bottom part both of which define a cavity within which plurality of hair fibers can be stored. The system further includes a cover portion wherein the cover portion is present at the top part of the elongated bottle. The system further includes a cap that is configured to be placed on the cover portion of the elongated bottle. The cover portion includes a plurality of openings, that have dimensions to allow at least one hair fiber. Further the plurality of hair fibers is released when the elongated bottle is stimulated. The top part of the elongated bottle is configured to be extended telescopically when rotated or pulled out due to which the surface area of touching hair fibers with the metal surface increase that removes static electricity from the hair.

According to yet another embodiment of the invention, there is provided a hair concealment method comprising pre-treating bald region of head that removes static electricity built up on the bald region and hence increases adhesive nature towards hair fiber. Further the method includes using a hair concealment device that comprises an elongated bottle that further includes a top and a bottom part both of which define a cavity within which plurality of hair fibers can be stored. The device further includes a cover portion wherein the cover portion is present at the top part of the elongated bottle. The device further includes a cap that is configured to be placed on the cover portion of the elongated bottle. The cover portion includes a plurality of openings, that have dimensions to allow at least one hair fiber. Further the plurality of hair fibers is released when the elongated bottle is stimulated. The top part of the elongated bottle is configured to be extended telescopically when rotated or pulled out due to which the surface area of touching hair fibers with the metal surface increase that removes static electricity from the hair. The method further includes providing a finishing treatment by spraying a soft fixing gel to affix the hair fibers to the bald region

Other and further aspects and features of the disclosure will be evident from reading the following detailed description of the embodiments, which are intended to illustrate, not limit, the present disclosure

### BRIEF DESCRIPTION OF THE DRAWINGS

The illustrated embodiments of the subject matter will be best understood by reference to the drawings, wherein like parts are designated by like numerals throughout. The following description is intended only by way of example, and simply illustrates certain selected embodiments of devices, systems, and processes that are consistent with the subject matter as claimed herein.

FIG. 1 illustrates a hair concealment system, in accordance with aspects of the embodiments;

FIG. 2A illustrates a hair concealment system with top part not extended, in accordance with other aspects of the embodiments;

FIG. 2B illustrates a hair concealment system with top part extended, in accordance with other aspects of the embodiments;

FIG. 3 illustrates a line diagram of a method of hair concealment, in accordance with aspects of the embodiments;

### DESCRIPTION

A few inventive aspects of the disclosed embodiments are explained in detail below with reference to the various figures. Embodiments are described to illustrate the disclosed subject matter, not to limit its scope, which is defined by the claims. Those of ordinary skill in the art will recognize a number of equivalent variations of the various features provided in the description that follows.

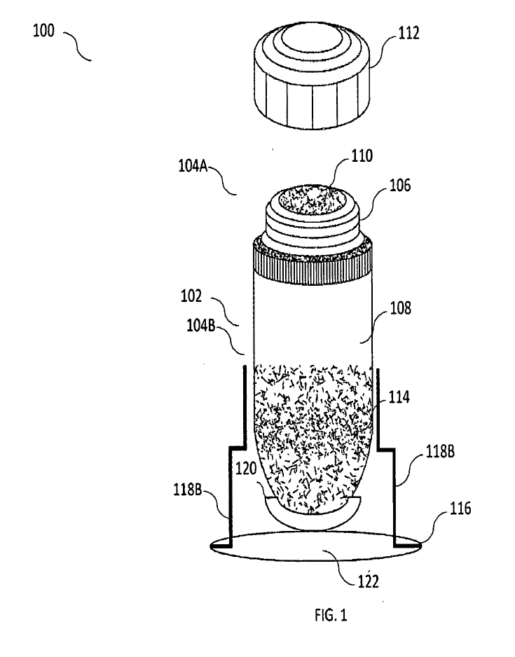

Now referring to FIG. 1 illustrating a hair concealment system 100 in accordance to an embodiment of the invention. The hair system 100, includes an elongated bottle 102. In an embodiment of the invention, the elongated bottle may be made up of plastic. The elongated bottle 102 further includes a top part 104A and a bottom part 104B wherein the top part 104A is releasable secured to the bottom part 104B. The top part 104A and the bottom part 104B together define a cavity 108. The cavity 108 stores a plurality of hair fibers 114 that can be natural, synthetic or a mixture of both. The top portion 104A includes a cover portion 106. The cover portion 106 includes threaded portion on which a cap 112 can be placed or fitted in a releasable manner. In other embodiments of the invention, the cap 112 may be placed on cover portion 106 by various other means like snap on features, other connectors etc.

Still referring to FIG. 1 the system cover portion 106 includes a plurality of openings 110 at its top part. The dimensions of the plurality of openings is such that they allow at least one hair fiber to fall through when the elongated bottle 102 is stimulated by shaking.

The system 100 further includes a metallic strip 120 that is placed at lowest end of the bottom part 104b. the metallic strip 120 has a small protruded extension, also of metal, that protrudes to inside of the cavity 108 from lowest position (not shown in figure). The protrusion helps in being in touch with the hair fibers 114. The system 100 further includes a metallic stand 116. The metallic stand includes a metallic base plate 122. Further, the metallic stand 116 includes a couple of receiving legs 118A and 118B. the receiving legs 118A-118B are configured to hold the elongated bottle 102 in a way such that the metallic strip 120 connects with the metallic base plate 122.

When the elongated bottle 102 is placed on the metallic stand 116, the metallic strip 120 touches the metallic base plate 122. Due to this since the protrusion is in contact with the hair fibers 114, it removes static electricity of the hair fibers when user removes the elongated bottle and touches any of metallic parts like receiving legs 118A-118B or metallic base plate 122 etc. This makes hair fibers affix more firmly on to a bald region of human head.

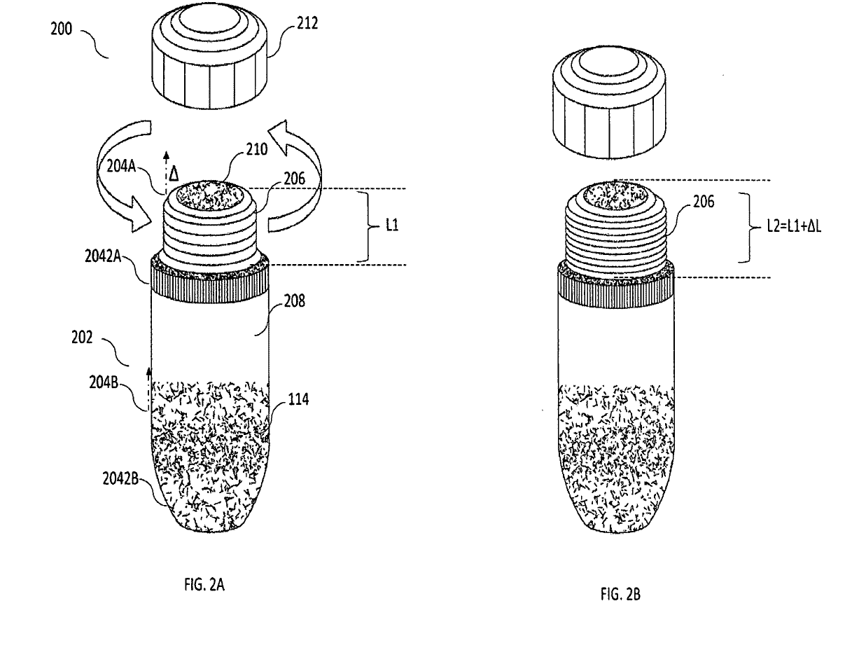

Now referring to FIG. 2A, a line diagram illustrating a hair concealment system 200 with top part 206 not extended, in accordance to an embodiment of the invention. The system 200 includes an elongated body 202. The elongated bottle may be made up of plastic. Further, the elongated body includes a top part 204A and a bottom part 204B. the top part 204A and bottom part 204B can be either attached to each other in a fixed manner or releasably secured to each other. The bottom part 240B also further includes a bottom portion 2042B and a top portion 2042A. The top portion 2042A may be made up of metal, so that when user uses the elongated bottle 202, his touch on the metal part removes static electricity of the hair fibers touching the top portion 2042A.

Still referring to FIG. 2A, the elongated body 202 further includes a cover portion, similar to the cover portion 106, to which a cap 212, similar to the cap 112, is releasably secured with the help of mating threads, snaps, or other connectors. The cap 212 is releasably securable to the elongated bottle 202 and covers a plurality of openings 210. The dimensions of the plurality of openings 210 is such that each of the plurality of openings allow at least one hair fibre through when the elongated bottle 212 is inverted and shaken.

Top part 204a is extendable outwards, that is it can be extended from its position, as displayed by FIG. 28. This can be achieved by either rotation of the top part 204A or by pulling out the top part 204A. Hence, in a normal position length of top part 204A is L1. However, after extension, length of the top part 204A is L2. Therefore L2=L1+ΔL wherein ΔL is length of extended part.

Since the top part 204A is made up of metal hence, its extension helps in increasing surface are of metal part due to which hair fibers touching the extended top portion 204A lose static electricity. The loss of static electricity is higher and more efficient.

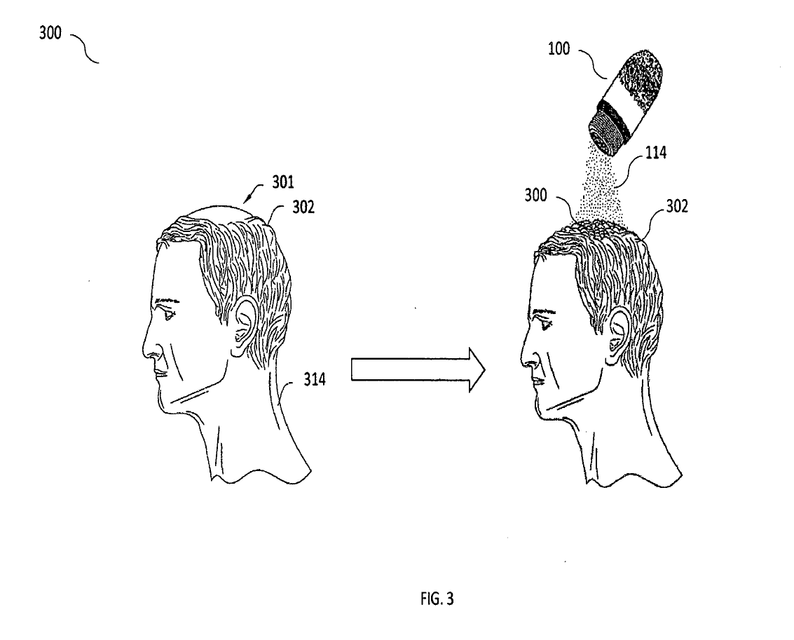

Now referring to FIG. 3, a line diagram illustrates a hair concealment method 300. The method 300 includes pre-treating a bald region 301 of a human 314. The bald region 301 may be surrounded by natural hair 302. The pre-treating can be done by rubbing hair with a cloth and then touching a metal plate. This pre-treating removes static electricity built on the bald region 301. Further in the method 300, the hair concealment device 100 or 200 (collectively referenced as 100) as described in above embodiments is utilized by a user. A user may shake the hair concealment device 100 over the bald region patch 301 such that the plurality of hair fibers 114. The plurality of hair fibers 114 are then secured to the existing hair 302 of the user 14 and conceal the alopecia patch 10 of the user 14.

The benefit of utilizing the hair concealment device 100 is that hair fibers 114 falling out, when the hair concealment device 100 is shaken, are without any static electricity. Hence, affixing property of hair fibers is improved because static electricity is removed from bald region 301 and even from the hair fibers 114. This step is then followed by a finishing treatment that is done by spraying of a soft gel. The spraying of the soft gel affixes the hair fibers 114 more firmly to the bald region 301.

The terminology used herein is for the purpose of describing particular embodiments only and is not intended to be limiting of the disclosure. It will be appreciated that several of the above-disclosed and other features and functions, or alternatives thereof, may be combined into other systems or applications. Various presently unforeseen or unanticipated alternatives, modifications, variations, or improvements therein may subsequently be made by those skilled in the art without departing from the scope of the present disclosure as encompassed by the following claims.

The claims, as originally presented and as they may be amended, encompass variations, alternatives, modifications, improvements, equivalents, and substantial equivalents of the embodiments and teachings disclosed herein, including those that are presently unforeseen or unappreciated, and that, for example, may arise from applicants/patentees and others.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

What is claimed is:

1. A hair concealment system comprising;
an elongated bottle, wherein the elongated bottle has a top and a bottom part, and wherein the top and the bottom part define an open cavity to store a plurality of hair fibers;
a cover portion, wherein the cover portion is present at the top part of the elongated bottle; and
a cap, configured to be placed on the cover portion of the elongated bottle, wherein the cover portion includes a plurality of openings, wherein each of the plurality of openings has a dimension to allow at least one hair fiber from the plurality of hair fibers to be released when the elongated bottle is stimulated.

2. The hair concealment system of claim 1, wherein the plurality of hair fibers is natural/real/human, or synthetic of mixture thereof.

3. The hair concealment system of claim 1, wherein the elongated bottle is made up of a plastic.

4. The hair concealment system of claim 1, further includes a thin metallic strip placed at the bottom of the elongated body.

5. The hair concealment system of claim of claim 4, further includes a metallic stand on which the elongated bottle is placed while not in use.

6. The hair concealment system of claim 5, wherein connection of the thin metallic strip with the metallic stand removes static electricity from the plurality of hair fibers.

7. A hair concealment system comprising;
an elongated bottle, wherein the elongated bottle has a top and a bottom part, wherein the top and the bottom part define an open cavity to store a plurality of hair fibers;
a cover portion, wherein the cover portion is present at the top part, wherein the cover portion includes a plurality of openings, wherein each of the plurality of openings has a dimension to allow at least one hair fiber from the plurality of hair fibers, to be released when the elongated bottle body is stimulated; and
wherein the top part is configured to extend outwards, wherein the static electricity of hair fibers touching the top part is removed.

8. The hair concealment system of claim 7, wherein the plurality of hair fibers is natural/real/human, or synthetic of mixture thereof.

9. The hair concealment device of claim 7, wherein the outward extension is through telescopic movement.

10. The hair concealment device of claim 7, wherein the bottom part is made up of a top portion and a bottom portion.

11. The hair concealment device of claim 10, wherein the top portion is made up of metal.

12. A hair concealment method comprising;
pre-treating a bald region of a human head, wherein the pre-treatment removes any static electricity from the bald region and increases its adhesive nature towards hair fiber;
using a hair concealment device, wherein the device comprises;
an elongated body, wherein the elongated bottle has a top and a bottom part, and an open cavity to store a plurality of hair fibers;
a cover portion, wherein the cover portion is present at the top part, wherein the cover portion includes a plurality of openings, wherein each of the plurality of openings has a dimension to allow at least one hair fiber from the plurality of hair fibers, to be released when the elongated bottle body is stimulated; and
wherein the top part is configured to extend outwards, wherein the static electricity of hair fibers touching the top part removes any static electricity;
providing a finishing treatment by spraying a soft gel to affix the hair fibers to the bald region.

13. The hair concealment method of claim 12, wherein the elongated bottle body is stimulated by shaking the bottle up and down.

14. The hair concealment method of claim 12, wherein the cover portion is facing towards the bald region. The hair concealment method of claim 12, wherein the plurality of hair fibers is natural, or synthetic of mixture thereof.

15. The hair concealment method of claim 12, wherein the pre-treating of the human head includes placing and removing a metal plate to remove static electricity.

16. The hair concealment method of claim 15, wherein the pre-treating further includes spraying a hair fixing mild gel to help fixation of the hair fibres.

17. The hair concealment method of claim 12, wherein the bottom part is made up of a top portion and a bottom portion.

18. The hair concealment method of claim 17, wherein the top portion is made up of metal.
